(19) United States
(12) Patent Application Publication
(10) Pub. No.: US 2023/0026471 A1
(43) Pub. Date: Jan. 26, 2023

(54) INJECTABLE MATERIALS DELIVERY DEVICES, SYSTEMS, AND METHODS

(71) Applicant: BOSTON SCIENTIFIC SCIMED, INC., MAPLE GROVE, MN (US)

(72) Inventors: Patrick A. Haverkost, Corcoran, MN (US); Joel N. Groff, Delano, MN (US); David Raab, Roseville, MN (US); Scott McGhee, Ottawa (CA); Mark Steven Smith, Coon Rapids, MN (US)

(73) Assignee: BOSTON SCIENTIFIC SCIMED, INC., MAPLE GROVE, MN (US)

(21) Appl. No.: 17/868,546

(22) Filed: Jul. 19, 2022

Related U.S. Application Data
(60) Provisional application No. 63/223,680, filed on Jul. 20, 2021.

(51) Int. Cl. A61M 5/142; A61M 5/158

(52) U.S. Cl. CPC A61M 5/1424; A61M 5/158

(57) ABSTRACT

A transport and delivery system which may be used for injectable materials such as radioactive injectables. The system includes a base configured to hold a material transport device containing the injectable material, and a cover including a material delivery system and a material access device. The cover is configured to be mated over the base to result in alignment of the material access device with the material transport device to allow the material delivery system to deliver the injectable materials from within the material transport device to a patient. A priming cap may be provided to facilitate priming of the material delivery system and material access device to be substantially airless.

### CROSS-REFERENCE TO RELATED APPLICATIONS

This application claims the benefit of priority under 35 U.S.C. § 119 to U.S. Provisional Application Ser. No. 63/223,680, filed Jul. 20, 2021, the disclosure of which is incorporated herein by reference in its entirety for all purposes.

### FIELD

The present disclosure relates generally to the field of devices, systems, and methods for delivering injectables such as for diagnostics, therapeutics, etc. More particularly, the present disclosure relates to the field of devices, systems, and methods for delivering injectables, such as for diagnostics, diagnostic imaging, therapeutics, etc. Even more particularly, the present disclosure relates to devices, systems, and methods for transporting, delivering, and injecting injectables, including radioactive or non-radioactive particles, beads, spheres, etc.

### BACKGROUND

Various containers and transport systems exist for pharmaceuticals, therapeutic devices, diagnostic devices, etc. to protect the pharmaceutical, therapeutic device, diagnostic device, etc., as well as the people handling the pharmaceutical, therapeutic device, diagnostic device, etc. Additional protective measures must be taken for certain pharmaceuticals, therapeutic devices, diagnostic devices, etc., such as radiopharmaceuticals, radiotherapeutic devices, radiodiagnostic devices, etc. In most cases local and global regulations dictate shielding requirements depending on the specific agent being transported. Radioactive materials may be harmful to the people handling such materials. Accordingly, the containers and transport systems must effectively shield the radioactive materials during transport from a pharmacy to patient treatment facility. Moreover, the containers and transport systems must effectively shield the radioactive materials from the medical professional handling the containers or transport systems (such as when diagnosing or treating a patient). In some instances, the materials which are transported/delivered are to be injected into a patient, and may be referenced herein as “injectables” and understood to include solid particles carried by a fluid medium, or fluids carried by a fluid medium. In addition, certain radiotherapeutic injectables include particles such as beads or bead-like particles which may have trouble passing from the container in which the radioactive material is provided through a microcatheter for administration to the patient. Improvements to devices, systems, and method for transporting and/or delivering injectables, such as from a pharmacy to a patient, would be welcome in the industry.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary.

In accordance with various principles of the present disclosure, a transport and delivery system includes a material transport device containing injectable material, a base configured to hold the material transport device, a material access device, and a cover configured to hold the material access device. In some aspects, the cover is configured to be positioned over the base to mate the cover with the base to cause the material access device to align with the material transport device to access the material transport device.

In some embodiments, the cover includes a material delivery system configured to deliver the injectable material to a patient. In some embodiments, the material delivery system includes the material access device, and a delivery conduit system fluidly coupled with the material access device. In some embodiments, the material access device includes at least one needle; and the delivery conduit system includes at least one tubing. In some embodiments, the at least one needle includes an inlet needle and an outlet needle; and the at least one tubing includes inlet tubing fluidly coupled with the inlet needle, and outlet tubing coupled with the outlet needle. In some embodiments, the material delivery system further includes a priming cap defining a sealed chamber therein and configured to cover sharp ends of the inlet needle and the outlet needle; and passing of flushing solution into the inlet tubing, through the inlet needle, through the sealed chamber, through the outlet needle, and through the outlet tubing results in a primed airless material delivery system primed for coupling with the base. In some embodiments, the material transport device includes a vial with a stopper defining a septum piercable by the at least one needle of the material access device as a result of mounting the cover on the base. In some embodiments, the at least one tubing includes an outlet tubing fluidly coupled with the at least one needle to deliver the injectable material to a patient; and the cover further includes a mounting arm configured to maintain the outlet tubing oriented at an angle below horizontal.

In some embodiments, the base is configured to permit ejection of the material transport device therefrom.

In accordance with various principles of the present disclosure, an airless primed material delivery system is provided. The system includes a material access device; a material conduit system fluidly coupled with the material access device, and a priming cap configured to cover the material access device and to maintain a sealed configuration of the material delivery system for priming.

In some embodiments, the material access device includes at least one needle; and the material conduit system includes at least one tubing. In some embodiments, the at least one needle includes an inlet needle and an outlet needle; the at least one tubing includes inlet tubing fluidly coupled with the inlet needle, and outlet tubing coupled with the outlet needle; the priming cap defines a sealed chamber therein configured to cover sharp ends of the inlet needle and the outlet needle; and passing of flushing solution into the inlet tubing, through the inlet needle, through the sealed chamber, through the outlet needle, and through the outlet tubing results in a primed airless material delivery system. In some embodiments, the system further including a cover holding the material access device and the material conduit system, the cover including a mounting arm configured to maintain the outlet tubing oriented at an angle below horizontal. The airless primed material delivery system further including a cover holding the material access device and the material conduit system, the cover being configured to align with a base holding a material transport device to align the material access device with the material transport device to deliver material from within the material transport device to a patient.

In accordance with various principles of the present disclosure, a method of transporting and delivering an injectable material includes delivering the injectable material in a material transport device; and placing a cover over the base configured to hold the material transport device; where the cover includes a material access device, so that upon mating the cover and the base, the material access device of the cover is aligned with the material delivery system held by the base to access the injectable material for delivery to a patient.

In some embodiments, the method further includes delivering the material delivery system separately from the base and cover. In some embodiments, the method further includes ejecting the material delivery system from the base when delivery of the injectable material to the patient is complete. In some embodiments, the cover further includes a material delivery system, and the method further includes priming the material delivery system to eliminate air within the material delivery system. In some embodiments, the material access device includes an inlet needle, an outlet needle, and a tubing system; and the material delivery system further includes a priming cap; wherein sharp ends of the inlet needle and the outlet needle extend into a chamber within the priming cap; and priming includes flushing a material through the inlet needle, through the chamber within the priming cap, through the outlet needle, and through the tubing system to eliminate air from within the material delivery system; and the method further includes retaining the material within the tubing system and removing the priming cap before mating the cover with the base to allow the sharp ends of the inlet needle and the outlet needle to extend into the material transport device to access the injectable material for delivery to a patient. In some embodiments, the method further includes delivering the material delivery system within the base to a medical facility at which the injectable material is to be delivered to the patient.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of a transport and delivery system formed in accordance with various principles of the present disclosure.

FIG. 2 illustrates a cross-sectional view of an example of an embodiment of a transport and delivery system, as in FIG. 1, along line II-II of FIG. 1.

FIG. 3 illustrates an exploded perspective view of an example of an embodiment of a transport device or container system for use with a transport and delivery system formed in accordance with various principles of the present disclosure.

FIG. 4 illustrates an exploded perspective view of an example of an embodiment of a delivery system formed in accordance with various principles of the present disclosure for use in a system such as illustrated in FIG. 1.

FIG. 5 illustrates a perspective view of another example of an embodiment of a needle cap/priming cap formed in accordance with various principles of the present disclosure.

FIG. 6 illustrates a perspective view of another example of an embodiment of a transport and delivery system formed in accordance with various principles of the present disclosure.

FIG. 7 illustrates a perspective view of another example of an embodiment of a transport and delivery system formed in accordance with various principles of the present disclosure.

FIG. 8 illustrates a perspective view of another example of an embodiment of a transport and delivery system formed in accordance with various principles of the present disclosure.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and “distal” refers to the direction or location furthest from the user, such as when using the device (e.g., introducing the device into a patient, or during implantation, positioning, or delivery). “Longitudinal” means extending along the longer or larger dimension of an element. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. It will be appreciated that “bore” is not limited to a circular cross-section. As used herein, a “free end” of an element is a terminal end at which such element does not extend beyond.

Various components and features of a transport and delivery system formed in accordance with various principles of the present disclosure may be used to transport and/or deliver a variety of injectable materials. It will be appreciated that reference herein is made to “injectable material” or “injectables” for the sake of convenience, and without intent to limit, to refer to materials in an injectable form, including, without limitation, pharmaceuticals, diagnostics (including, without limitation, imaging materials), therapeutic devices (including, without limitation, particles, beads, spheres, etc.), etc., and including solid particles carried by a fluid medium and/or fluids carried by a fluid medium. Reference may be made herein to radioactive injectable materials (e.g., radiopharmaceuticals, radiotherapeutics, radiodiagnostics, radioimaging, tracers, etc.), as components and features of the disclosure are suitable for such materials, although the disclosure is not limited in this context. For instance, a transport and delivery system formed in accordance with various principles of the present disclosure may be used in conjunction with transport and/or delivery of TheraSphere™ Y-90 Glass Microspheres (glass microspheres infused with Yttrium, particularly Y-90), sold by Boston Scientific Corporation, for cancer treatment (radiotherapy). A transport and delivery system formed in accordance with various principles of the present disclosure may likewise be usable with diagnostics, such as radiodiagnostics and/or radioimaging, which may be performed in conjunction with a therapeutic course. For instance, beads or microbeads or microspheres (such terms may be used interchangeably herein, reference generally being made to microspheres for the sake of convenience and without intent to limit) may be provided with a radioactive element for imaging and diagnostic purposes. More particularly, microspheres, which may be formed of a suitable material such as glass, may be infused or labelled or coated with radioactive materials, such as Technetium, particularly Tc-99m. The Tc-99m labelled microspheres may function as a surrogate or proxy for the TheraSpheres™, as the Tc-99m microspheres have substantially the same flow characteristics and therefore can be used for imaging and diagnostics before injection of TheraSpheres™ which have a significantly longer half-life (Y-90 has a half-life of 64 hours in contrast with Tc-99m, which has a half-life of 6 hours).

Injectable materials to be delivered to a patient in accordance with various principles of the present disclosure are provided in a vessel such as a vial or a syringe or the like. Reference is made herein to a vial for the sake of convenience and without intent to limit. The delivery may be described herein for the sake of convenience as at a point of administration, or the like, without intent to limit, such as a medical facility, an interventional radiology (“IR”) suite, etc. In some embodiments, the injectable material is provided in a vial which may be formed of glass or any other suitable material for securely containing the injectable material. The injectable material may be a liquid, a solid (e.g., a dry solid, such as microspheres, typically suspended/carried in a fluid), or combinations thereof (e.g., microspheres in deionized water, microspheres with a fluoroscopic contrast medium, etc.). A stopper may close or seal the open end of the vial and may include a septum (formed separately from or as a part of the stopper) which may be capable of being pierced and preferably self-sealing (e.g., formed of an elastomeric material) to access the injectable material for delivery to a patient. The injectable material may be used for diagnostics, imaging, treatment, therapy, etc., without limitation, the present disclosure not being limited in this context. Moreover, the injectable material may be in any of a variety of desired or indicated forms, such as a fluid (e.g., a typical pharmaceutical form), a gel, or a solid (e.g., solid particles such as beads, microbeads, microspheres, or other particles, such as known for therapeutical use, etc., typically suspended/carried in a fluid).

In accordance with one aspect of the present disclosure, a transport and delivery system includes a material transport device which includes at least one container for the vial, particularly if the injectable material is radioactive. The at least one container may include a first container in which the vial is held. The first container may alternately be referenced as a shield, liner, inner container, etc., and such terms may be used interchangeably herein without intent to limit. The first container may be formed of a radiation shielding material capable of blocking any radioactive particles (e.g., beta or gamma radiation) from passing therethrough from the vial. Typical radiation shielding materials include lead or tungsten or stainless steel or plastic (e.g., acrylics) or combinations thereof or composite materials containing such elements (e.g., lead-infused polycarbonate, tungsten-infused polycarbonate, etc.). It will be appreciated that exposure time, distance, and shielding material efficiency are factors considered for radiation safety requirements, such as based on which radioactive isotope is being handled or utilized. The at least one container may further include a second container in which the first container, with the vial therein, may be positioned, held, placed, carried, transported, etc. (such terms and the like being used interchangeably herein without intent to limit). The second container may alternately be referenced as an outer container, outer shell, shell, etc., and such terms may be used interchangeably herein without intent to limit. The second container may be formed of any suitable material with a suitable thickness capable of protecting the first container and the vial, as may be appreciated by those of ordinary skill in the art. It will be appreciated that the material of the second container need not be a material which shields against radioactivity, and may simply be a polymeric material such as a cellulose-based material which is biodegradable (e.g., polystyrene) or other plastic or metal (e.g., stainless steel) material. The at least one container may be a radiopharmaceutical pig, such as known for carrying radioactive materials.

In accordance with one aspect of the present disclosure, a transport and delivery system includes a material transport system including the material transport device and a base for the material transport device. In some embodiments, the base may be configured as a carrying case and may include a base unit in which the material transport device is held, and a lid covering the base. The lid may include a handle to facilitate handling (e.g., carrying) of the material transport system. Any of a variety of securement devices may be used to hold the lid in place with respect to the base unit. For instance, bands (e.g., elastic straps), latches, mating configurations (detents and grooves, threads, etc.), clamps, vises, etc. may be used to hold the lid with respect to the base unit. In other embodiments, the material transport device is transported separately and then positioned in a base at the point of administration of the injectable material contained within the vial in the material transport device. In such embodiment, the base may be configured to securely (and safely, if the material is radioactive) hold the material transport device therein, and/or to stabilize the material transport device during delivery of the injectable material contained therein to the patient.

In accordance with various principles of the present disclosure, a base configured to hold a material transport device therein is configured to mate with a material delivery system. More particularly, the base may be configured to align a material delivery system with the material transport device to facilitate delivery of the injectable material contained within the material transport device to a patient. The base may be configured as part of a material transport system, or may be formed separately therefrom and configured to receive a material transport device therein. The base may be in any of a variety of shapes and/or configurations such as to facilitate holding and/or to facilitate stability when set on a support surface. In some embodiments, at least a portion of the base is shaped, sized, dimensioned, and configured to mate with a material delivery system to be coupled therewith. Coupling of the material delivery system with the base allows for the material delivery system to access the injectable material within the material transport device held within the base.

A material delivery system formed in accordance with various principles of the present disclosure may include a material access and delivery system for accessing and delivering the materials within the material transport device to the patient. In some embodiments, the material access and delivery system includes at least one material access device such as a needle. The needle may be configured to pierce the septum of a vial containing the injectable materials. In some embodiments, the material access and delivery system includes a cover which may be configured to hold components of the system. In accordance with various principles of the present disclosure, a base of a transport and delivery system and the cover of the material access and delivery system are configured to facilitate alignment of the at least one material access device with the material transport device to access the injectable materials within the material transport device. Alignment of the material access device with the material transport device may be achieved substantially automatically upon mating the cover over the base. Upon closing the cover and the base together, the material access device substantially automatically extends into (e.g., pierces the septum of the vial of) the material transport device to access the injectable material therein for delivery to a patient. Automatic alignment of the material delivery system, and particularly a material access device thereof, with the material transport device provides various benefits, such as, without limitation, facilitating set up for delivery of injectables to the patient, reducing the amount of time required to convey the injectable material to the material delivery system, reducing the amount of handling of the material transport device by the medical professional (which may be particularly of interest if the injectable material is radioactive), providing a compact contained system, etc.

In some embodiments, the material access and delivery system includes delivery tubing or other delivery conduit(s) (for the sake of convenience, reference is made herein simply to delivery tubing, without intent to limit) through which the injectable material is conveyed to the patient. Delivery tubing may be coupled directly or indirectly to other components of the material delivery system as may be readily appreciated by those of ordinary skill in the art. The material delivery system may further include a material impelling system configured to draw, convey, impel, or otherwise direct (such terms being used interchangeably herein without intent to limit) the materials from within the material transport device into the delivery tubing. For instance, the material access device may include a first needle and a second needle, inlet tubing coupled to the first needle (which may be alternately referenced as an inlet needle without intent to limit), and outlet tubing connected to the second needle (which may be alternately referenced as an outlet needle without intent to limit). Sterile injectable fluid may be injected via the inlet tubing into the first needle to push material within the material transport device into the second needle and into the outlet tubing (which may alternatively be referenced as delivery tubing). If the injectable material is in the form of solid particles, a liquid such as saline (e.g., sterile saline) or other fluids approved for intravenous or intraarterial injection may be injected to fluidize the solid particles, not only to deliver the solid particles from the material transport device but also to facilitate flow of the solid particles into the patient. In some embodiments, the material delivery system is configured to maintain the delivery tubing oriented to facilitate flow of the solid particles into a microcatheter for injection into the patient. For instance, a catheter mounting arm or other structure (e.g., strut) configured to support or hold the outlet tubing in a downward orientation (negative angle from horizontal) towards the patient may be associated with the delivery system to facilitate flow of the solid particles and to prevent the solid particles from catching or otherwise becoming stuck, such as within the outlet tubing and/or manifold coupling the delivery tubing to the microcatheter, etc.

In accordance with various principles of the present disclosure, the material delivery system may be configured to be primed prior to being coupled with the material transport device to deliver the injectable material to the patient. Such priming may be particularly advantageous to create a substantially airless primed delivery system. It will be appreciated that it is particularly desirable to eliminate any air pockets in a radioactive injectable which may cause the radioactive materials to be misdirected and possibly harm unintended target tissue within the patient. In accordance with one aspect, a priming tool may be provided to facilitate priming of the material delivery system. In some embodiments, the priming tool may also be configured to cover (e.g., to protect) the sharp end of the one or more needles of the material delivery system. In such embodiment, the priming tool covers the sharp ends of a first needle and a second needle. For instance, the priming tool may comprise a cap with a septum, the first and second needle piercing through the septum to be contained within a holding area within the priming tool. Such holding area may be substantially sealed. Accordingly, priming fluid may be injected into inlet tubing, through the first needle and into the holding area within the priming tool, from the holding area into the second needle, and then into outlet tubing. Flushing the material delivery system in such manner eliminates air and any other desired materials within the material delivery system. Once a steady stream of priming fluid is achieved through the material delivery system, or priming has been performed satisfactorily for a selected amount of time or until another indication of sufficient priming is reached (e.g., lack of visible air bubbles in the tubing), the inlet tubing and the outlet tubing may be closed/sealed, such as with one-way valve or other closure, until the medical professional is ready to couple the material delivery system with the material transport device. A one-way valve generally may be provided on the inlet tubing, such that active closing of the delivery tubing is not necessary to retain the flushing/priming fluid therein. If a one-way valve is not be suitable for use with outlet tubing (e.g., if the injectable material includes particles which may catch or otherwise collect along a one-way valve and not be delivered to the patient), a finger clip or a cap (e.g., tethered to the outlet tubing) or another structure for pinching or closing the outlet tubing may be provided on the outlet tubing to maintain the material delivery system as an airless primed system.

Various embodiments of a transport and delivery system will now be described with reference to examples illustrated in the accompanying drawings. Various features and structures disclosed with respect to an example of an embodiment should be understood as being separate and independent from one another and combinable with one another unless otherwise indicated. Reference in this specification to “one embodiment,” “an embodiment,” “some embodiments”, “other embodiments”, etc. indicates that one or more particular features, structures, and/or characteristics in accordance with principles of the present disclosure may be included in connection with the embodiment. However, such references do not necessarily mean that all embodiments include the particular features, structures, and/or characteristics, or that an embodiment includes all features, structures, and/or characteristics. Some embodiments may include one or more such features, structures, and/or characteristics, in various combinations thereof. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. When particular features, structures, and/or characteristics are described in connection with one embodiment, it should be understood that such features, structures, and/or characteristics may also be used in connection with other embodiments whether or not explicitly described, unless clearly stated to the contrary. It should further be understood that such features, structures, and/or characteristics may be used or present singly or in various combinations with one another to create alternative embodiments which are considered part of the present disclosure, as it would be too cumbersome to describe all of the numerous possible combinations and subcombinations of features, structures, and/or characteristics. Moreover, various features, structures, and/or characteristics are described which may be exhibited by some embodiments and not by others. Similarly, various features, structures, and/or characteristics or requirements are described which may be features, structures, and/or characteristics or requirements for some embodiments but may not be features, structures, and/or characteristics or requirements for other embodiments. Therefore, the present disclosure is not limited to only the embodiments specifically described herein.

It will be appreciated that common features among the various drawings are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments are generally designated with the same reference numbers increased by a multiple of 100 and redundant description is generally omitted for the sake of brevity. Moreover, certain features in one embodiment may be used across different embodiments and are not necessarily individually labeled when appearing in different embodiments.

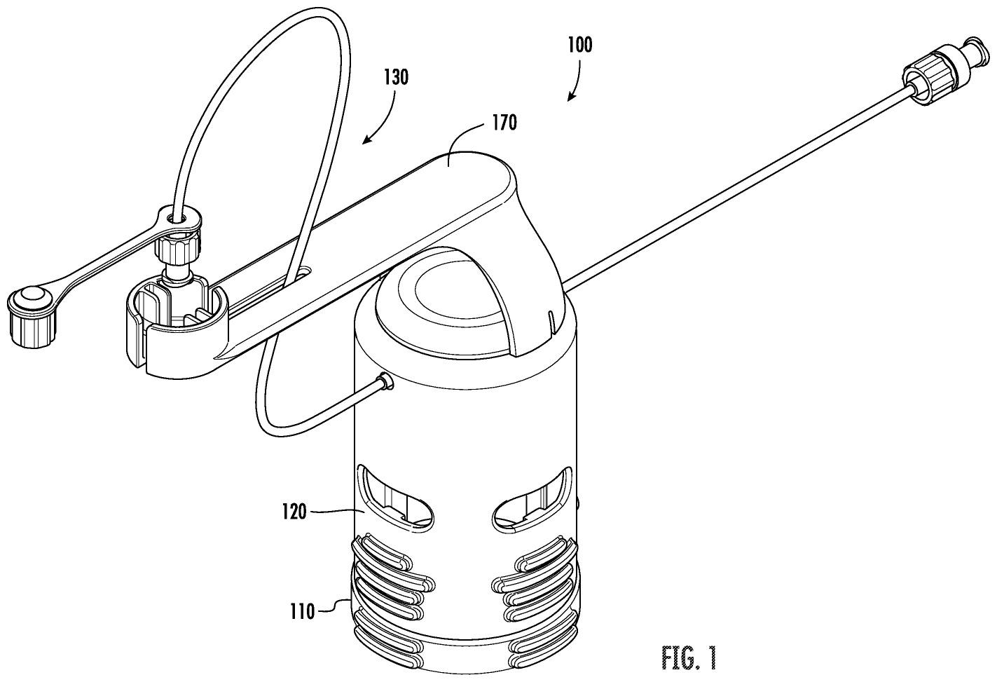

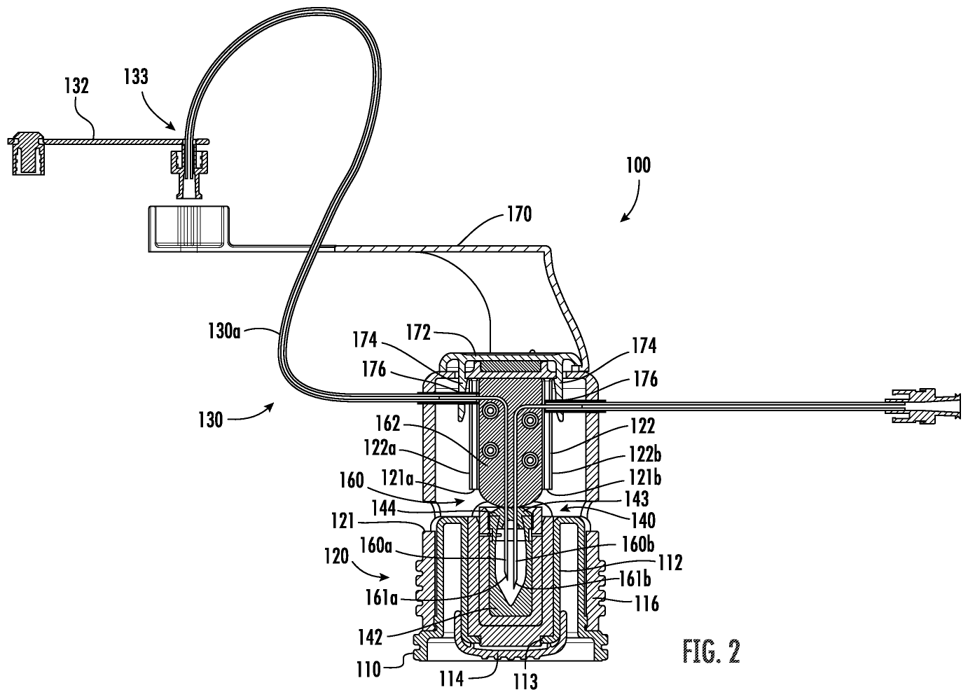

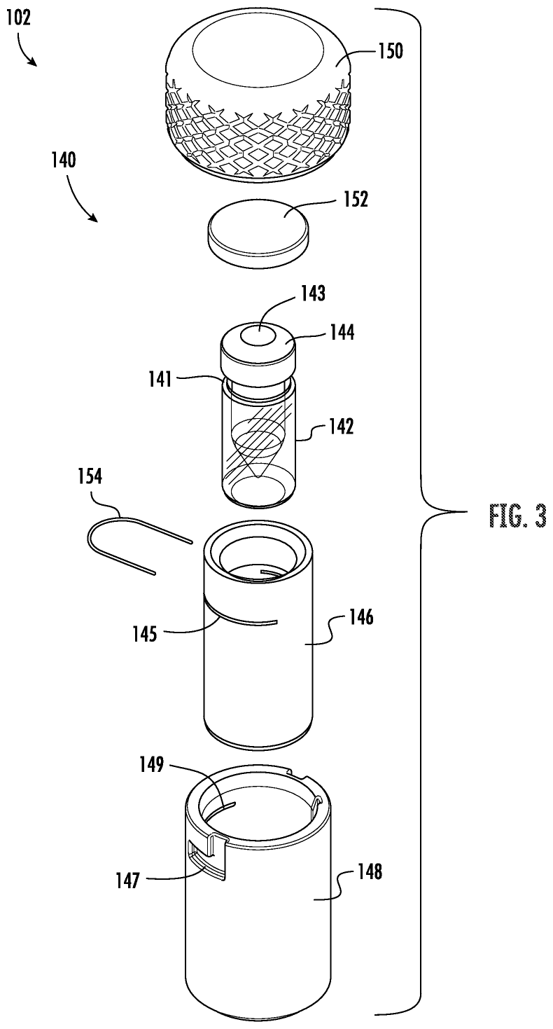

Turning now to the drawings, an example of an embodiment of a transport and delivery system 100 formed in accordance with various principles of the present disclosure is illustrated in FIG. 1. The illustrated transport and delivery system 100 includes a base 110 and a cover 120, and a delivery tubing system 130 associated therewith. A material transport device 140 (shown in FIG. 2, and in further detail in FIG. 3) is provided within the transport and delivery system 100 and contains injectable material to be conveyed through the tubing system 130. The base 110 may be configured to hold therein the material transport device 140, as may be appreciated with reference to FIG. 2. The base 110 may also be configured to mate with the cover 120 to facilitate coupling of the tubing system 130 with the material transport device 140, such as to essentially automatically couple the tubing system 130 with the material transport device 140 upon completion of placement of the cover 120 over the base 110.

An example of an embodiment of a material transport device 140 is illustrated in further detail in FIG. 3. The material transport device 140 may be considered an assembly or part of a material transport system 102 with multiple components including, at least a vessel (which may be referenced herein as a vial for the sake of convenience and without intent to limit) in which injectable material is contained. Additionally, the material transport device 140/material transport system 102 includes one or more protective containers for the vessel. If the injectable material is radioactive, at least one of the protective containers is a radioactive shield to block radioactive emissions from the vessel. In the example of an embodiment illustrated in FIG. 2, the material transport device 140 includes a vial 142 (which may be formed of glass) closed with a stopper 144 for containing the injectable material. The stopper may be formed of an elastomeric material (e.g., chlorobutyl rubber), so that a portion of the stopper 144 may be configured as a septum 143 which may be pierced (e.g., by a needle) to access and to deliver the injectable material from within the vial 142. Alternatively, the septum 143 may be formed as a separate element (e.g., with the stopper including a rigid ring holding an elastomeric septum). The material transport device 140 may further include a first container 146 in which the vial 142 may be positioned. The first container 146 may be formed of a material which blocks radioactive emissions (e.g., beta or gamma particles), such as lead, tungsten, or stainless steel, and therefore may be considered a radioactive shield for the vial 142 if the injectable material therein is radioactive. The first container 146 may be placed within a second container 148, such as to further protect the vial 142 as well as the first container 146. The second container 148 may be formed from a polymer (generally more lightweight than the material of the first container 146), such as polystyrene and may be considered a shell.

In the example of an embodiment illustrated in FIG. 3, a protective cap 150 is provided to hold the vial 142 within the first container 146, and, if a second container 148 is provided, to hold the vial 142 and first container 146 within the second container 148. The protective cap 150 need not be made of a radiation shielding material, as the shield provided by the first container 146 may be sufficient if the vial 142 does not extend beyond the top of the first container 146. For instance, the protective cap 150 may be formed of a polymeric/plastic material. If desired, a sheet 152 of radiation shielding material (e.g., a disk, such as a ⅛″ (0.3175 cm) disk of stainless steel) may be inserted within the protective cap 150. The protective cap 150 and the second container 148 (or the first container 146 if a second container 148 is not included) may be configured to be coupled together so that the protective cap 150 remains in place. For instance, a locking groove 147 (e.g., an L-shaped groove such as used with Luer locks) may be provided on an exterior of the second container 148 engageable with a mating detent (not shown, but any suitable configuration as may be appreciated by one of ordinary skill in the art to mate with the locking groove 147) within the protective cap 150. Other configurations, such as threads, a friction fit, etc., are within the scope of the present disclosure.

In addition, in the example of an embodiment illustrated in FIG. 2 an additional holding element 154 may be provided to hold the vial 142 within the first container 146. The holding element 154 may be in the form of a metal wire or other low profile element configured to restrain the vial 142 from movement with respect to the first container 146. As illustrated, the holding element 154 is insertable between the stopper 144 of the vial 142 and a shoulder 141 of the vial 142 below the stopper 144, and held with respect to the first container 146 by extending through an opening 145 in the first container 146. The holding element 154 may further be held with respect to the second container 148 by extending within a groove 149 within the interior of the second container 148. In the example of an embodiment illustrated in FIG. 2, the holding element 154 is a U-shaped wire with the cross-portion (extending between the legs of the U-shape) extending into the groove 149 in the second container 148.

The material transport device 140 may be transported in a carrying case to the site at which the injectable material is to be delivered to a patient (e.g., at a point of administration). If the injectable material is radioactive, then the carrying case is formed in compliance with regulations known in the industry for protecting against radioactive emissions therefrom.

Once the material transport device 140 is at the medical facility, the material transport device 140 is placed in a holder 112 within the base 110, as may be appreciated with reference to FIG. 2. The holder 112 may be sized, shaped, configured, and dimensioned to securely contain the material transport device 140 therein. For instance, it generally is desirable that the material transport device 140 is substantially immovable (e.g., can move less than at most about 0.1 mm) when within the holder 112. Additionally, the holder may be configured to facilitate removal of the material transport device 140 therefrom once the injectable material has been delivered to the patient. In the example of an embodiment illustrated in FIG. 1, an opening 113 is defined along the bottom the holder 112 through which a portion of the bottom of the material transport device 140 is accessible. The medical professional may access the bottom of the material transport device 140 through the opening 113 to push the material transport device 140 out of the holder 112. A holder cover 114, such as a rubber base, may be positioned over the opening 113 and may be sufficiently flexible to be pressed to push the material transport device 140 to eject the material transport device 140 out of the holder 112 (such as to allow insertion of another material transport device 140). The holder cover 114 may also be suitable as a sterile barrier for the material transport device 140.

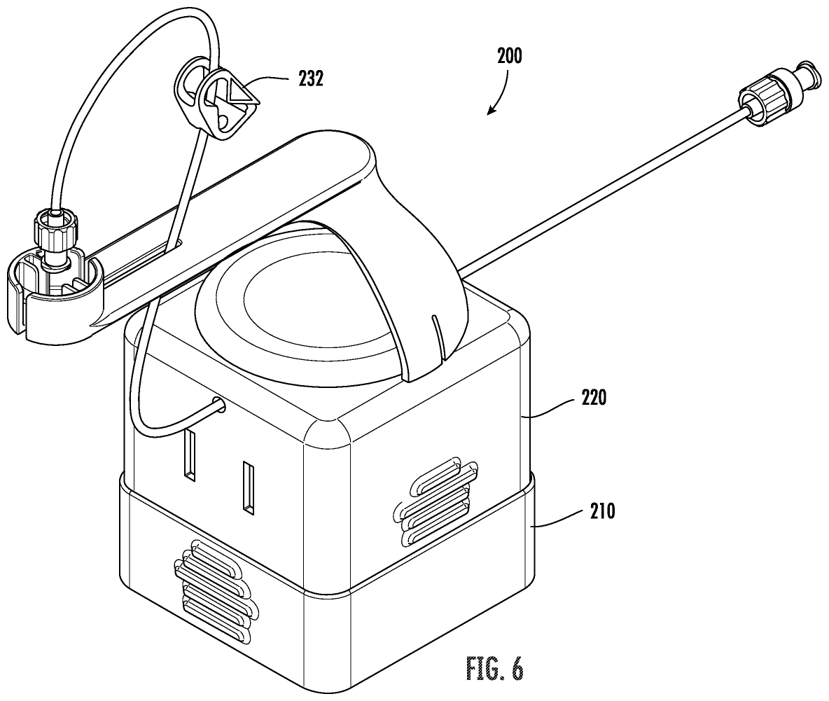

The base 110 may include an outer wall 116 to define a desired outer dimension and/or configuration of the base 110. For instance, in some embodiments, it may be desirable for the base 110 to be manually held by the medical professional administering the injectable material from within the material transport device 140 to the patient. The outer wall 116 may be sized, shaped, dimensioned, and configured to facilitate grasping within a medical professional's hand, such as once mated with the cover 120. For instance, the outer wall 116 may be round or rounded to facilitate grasping, such as in the example of an embodiment illustrated in FIG. 1. In other embodiments, such as illustrated in FIGS. 6-8 (and described in further detail below), it may be desirable for the base 110 to be set on a support surface, with the base 110 being sufficiently sized, shaped, dimensioned, and configured to provide sufficiently stability so the transport and delivery system 100 remains upright independently and is resistant to being knocked over or otherwise destabilized.

As noted above, in accordance with various principles of the present disclosure, the base 110 may be configured to mate with the cover 120. In the example of an embodiment illustrated in FIG. 1 and FIG. 2, the outer wall 116 of the base 110 may be configured to mate with (e.g., receive) the cover 120 by having similar shapes. Additionally or alternatively, the base 110 and the cover 120 may have engagement features for holding the base 110 and cover 120 in place with respect to each other. For instance, mating ribs, mating grooves and detents, or other engagement features may be provided on the base 110 and cover 120. In accordance with one aspect of the present disclosure, the cover 120 is mounted over the base 110 by being positioned over and being moved down onto the base 110. In such configuration, engagement features (e.g., axial ribs and/or engaging detents, shoulders, grooves, recesses, etc. as known to those of ordinary skill in the art, the present disclosure not being limited by a specific configuration) facilitate sliding axial movement between the base 110 and cover 120 to be mated together, and to be held together once positioned in their final positions with respect to each other, such as illustrated in FIG. 1 and FIG. 2.

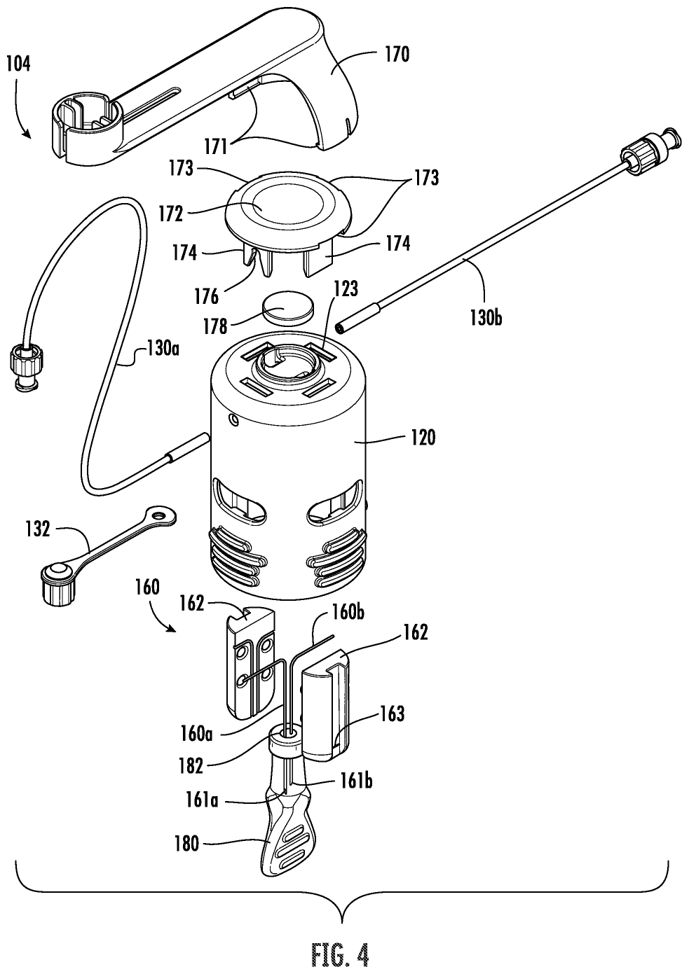

As noted above, the base 110 and the cover 120 may be configured such that upon engagement of the cover 120 with the base 110, the tubing system 130 is fluidly coupled with the material transport device 140 configured to deliver injectable material from within the vial 142 to a patient, as may be appreciated with reference to FIG. 2. More particularly, mounting and engagement of the cover 120 with the base 110 aligns a material access device 160 within the cover 120 with the vial 142 in which the injectable material is contained. The material access device 160 illustrated in FIG. 2 includes a needle (more particularly, a pair of needles 160a, 160b), or any other type of tubular element configured to access and convey the injectable material from the vial 142. The material access device 160 may include a material access device clamshell 162 configured to hold the needles 160a, 160b. In some embodiments, the needles 160a, 160b are held in offset positions, such as to create a vortex upon priming the material delivery system (described in further detail below) and/or delivering injectable material. The material access device clamshell 122 may be carried by a material access device holder 122 formed within the cover 120. In the example of an embodiment illustrated in FIG. 2, the material access device holder 122 includes at least two spring arms 122a, 122b configured to engage or mate with the exterior of the material access device clamshell 162. For instance, spring arms, similar to spring arms 122a, 122b, may be provided with detents or shoulders or may be otherwise formed (such as at the free ends 121a, 121b thereof) to lock into or below grooves or shoulders in the material access device clamshell 162 (see, e.g., shoulders 163 in FIG. 4) in a manner which may be appreciated by those of ordinary skill in the art. A window 121 may be provided in the cover 120 or at least portions of cover 120 may be formed of a transparent/translucent material to allow viewing of the material access device 160 and the material transportation device 140 such as to view alignment and/or to assure there are no leaks. Movement of the cover 120 towards the base 110 causes the material access device 160, particularly, the sharp ends 161a, 161b of the needles 160a, 160b of the material access device 160, to pierce the septum 143 of the stopper 144 to access the injectable material within the vial 142. The delivery tubing 130a of the tubing system 130 is fluidly coupled with the material access device 160 (in particular, with the delivery needle 160a) so that once the material access device 160 is fluidly coupled with the interior of the vial 142, the injectable material may be delivered to the delivery tubing 130a for delivery to a patient.

If the injectable material is in the form of microspheres or another form which may not flow readily through and/or out of the delivery tubing 130a (which may alternatively be referenced as outlet tubing without intent to limit), a mounting arm 170 may be provided on the cover 120, as illustrated in FIG. 1 and FIG. 2. The mounting arm 170 may be a strut or other structure configured to maintain the delivery tubing 130a in a desired orientation. For example, the mounting arm 170 may be configured to hold the delivery end 133 of the delivery tubing 130a in a downward, nonhorizontal orientation (e.g., at least about −35° with respect to a horizontal plane, or at least about −45° with respect to a horizontal plane, up to an approximately vertical orientation, including increments of −1° from a −30° orientation to an approximately −90° orientation). Such orientation is selected so that the injectable material readily passes out of the material delivery system (e.g., out of the delivery tubing 130a) and into the patient, leaving minimal, if any, residue behind in the delivery tubing 130a. The mounting arm 170 may be formed as a separate element coupled (e.g., snapped or otherwise mated with) the outer wall 126 of the cover 120. The mounting arm 170 may be in other forms or configurations than that illustrated in FIGS. 1 and 2, such as, without limitation, the form/configuration illustrated in the examples of embodiments a transport and delivery systems illustrated in FIGS. 6-8. In the example of an embodiment illustrated in FIG. 1, FIG. 2 and FIG. 4, the mounting arm 170 is mounted to the cover 120 via a mounting cap 172 with mating features (e.g., clips 171 on the mounting arm 170 fitting with slots 173 in the mounting cap 172 or other suitable mating features as may be appreciated by those of ordinary skill in the art). The mating features may be configured to align the mounting arm 170 to be positioned for holding the delivery tubing 130a (in contrast with the inlet tubing 130b) in the desired orientation. Additionally, the mounting cap 172 may include mating features to facilitate coupling with the cover 120. In the example of an embodiment illustrated in FIG. 2 and FIG. 4, the mounting cap 172 includes mounting legs 174 extending through mounting slots 123 in the cover 120. Tube guides 176 may be formed in ends of the mounting legs 174 of the mounting cap 172 to hold the tubing system 130 in place within the cover 120, as may be appreciated with reference to FIG. 2 and FIG. 4. Optionally, a sheet 178 of radiation shielding material (e.g., a disk, such as a ⅛″ (0.3175 cm) disk of stainless steel) may be provided beneath the mounting cap 172.

In accordance with various principles of the present disclosure, at least the cover 120 along with tubing system 130 and the material access device 160 may be considered a part of a material delivery system 104. Before mating of the cover 120 with the base 110, the material delivery system 104 may be primed so that the material delivery system 104 is an airless primed system with the associated benefits known to those of ordinary skill in the art of not injecting air bubbles or pockets in the injectable material during delivery once assembled with the base 110.

Components of an example of an embodiment of a material delivery system 104 of a transport and delivery system 100 such as illustrated in FIG. 2 are illustrated in an exploded configuration in FIG. 4, with a priming cap 180 (shown partially in phantom). The priming cap 180 is configured to cover the sharp ends 161a, 161b of the needles 160a, 160b of the material access device 160 (e.g., to protect against injury, accidents, inadvertent piercing, etc.) before accessing the contents of the vial 142 (see, e.g., FIG. 2) in which the injectable material is contained. Additionally, the priming cap 180 may include a septum 182 (which may be a part of the priming cap 180 or a separate element) through which the needles 160a, 160b extend. A sealed cavity or chamber may be defined within the priming cap 180, sealed by the septum 182. Once the patient and medical professional are ready for administration of the injectable material to the patient (and the material transport device 140 is ready within the base 110), the material delivery system 104 may be primed. A flushing solution, such as sterile saline, may be injected into the inlet tubing 130b of the tubing system 130 to flow into the inlet needle 160b, through the cavity created within the priming cap 180, out the outlet needle 160b, and out the delivery tubing 130a. Once the material delivery system 104 is sufficiently primed, a tethered cap 132 (such as illustrated in FIG. 1 and FIG. 4) or finger clip 232 (such as illustrated in FIG. 6) or other closure element (e.g., a pinching clip or other closure element known to those of ordinary skill in the art) mounted on the delivery tubing 130a or formed on the mounting arm 170, in conjunction with a one-way valve in the inlet tubing 130b may be actuated to maintain the material delivery system 104 as an airless primed system. The material delivery system 104 may thereby be fluid locked on both ends so that air cannot enter the system. The protective cap 150 of the material transport device 140 and the priming cap 180 over the needles 160a, 160b may be removed, and the cover 120 may be positioned over and pushed onto the base 110. As described above, the material access device 160 aligns with the material transport device 140 so that the needles 160a, 160b pierce the septum 143 of the stopper 144 on the vial 142 to access the injectable material within the vial. Tactile or audible feedback confirm mating alignment.

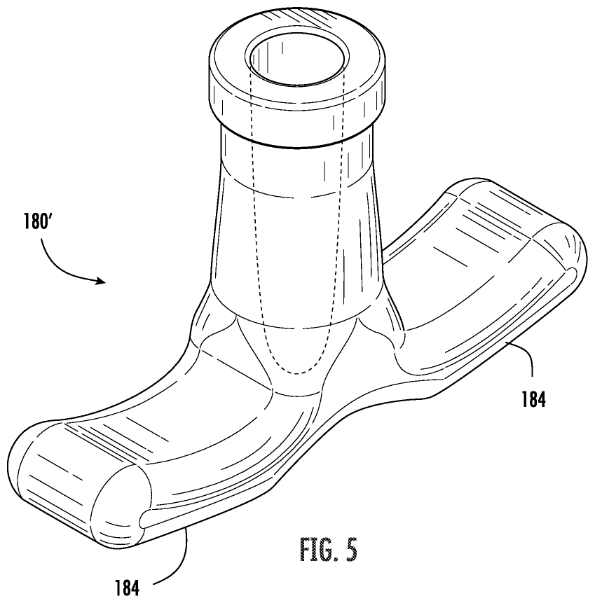

It will be appreciated that various modifications to forms or configurations of a transport and delivery system formed in accordance with various principles of the present disclosure are within the scope and spirit of the present disclosure. For instance, as noted above, the transport and delivery system may be sized, shaped, dimensioned, and configured to stably positioned on a support surface. If material delivery system is larger than the material delivery system 104 of FIGS. 1, 2, and 4 (with a generally rounded cross-sectional shape), various modifications may be made. For instance, a modified priming cap 180′ may include finger grips 184 extending outwardly therefrom, such as in the example of an embodiment illustrated (partially in phantom) in FIG. 5. Alternatively, the priming cap may simply be in the form of protective tubing over the sharp ends of the needles of the material access device 160.

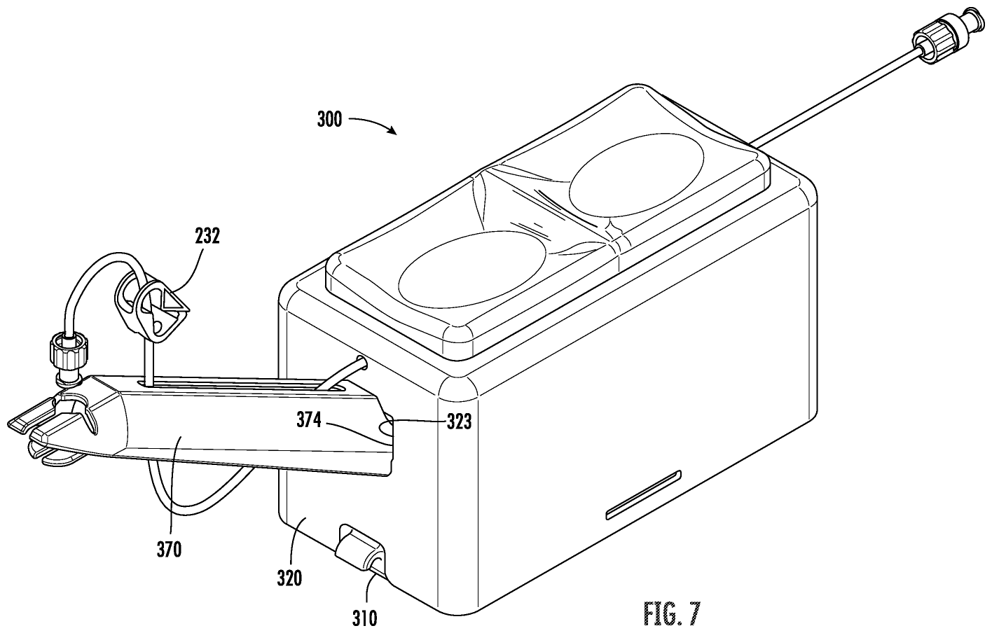

Additionally, various modifications to the cross-sectional shapes of the bases and covers of the transport and delivery system may be made, such as in the examples of embodiments illustrated in FIGS. 6-8. In the example of an embodiment of a transport and delivery system 200 illustrated in FIG. 6, the base 210 and the cover 220 each have substantially square cross-sectional shapes. In the example of an embodiment of a transport and delivery system 300 illustrated in FIG. 7, the base 310 and the cover 320 each have substantially square cross-sectional shapes. The internal configurations of the transport and delivery systems 200 and 300 of FIGS. 6 and 7, respectively, may be substantially similar to the internal configuration of the transport and delivery system 100 of FIGS. 1, 2, and 4 (with modifications to shape and size and dimension as may be readily appreciated).

A cover with a cross-sectional shape may allow for mounting of a mounting arm 370 along a side thereof, such as illustrated in FIG. 7, rather than along the top side thereof (as in the examples of embodiments illustrated in FIGS. 1, 2, 4, and 6). It will be appreciated that the mounting arm 370 may include a mounting leg 374 (mostly within and obstructed from view by the cover 320) which extends within a mounting slot 323 in the cover 320, similar to the mounting structure of the example of embodiment illustrated in FIGS. 1, 2, and 4. Otherwise, various components, features, etc. may be similar to those described above with respect to the transport and delivery system 100 illustrated in FIGS. 1, 3, and 4, and are not described further herein for the sake of brevity and without intent to limit.

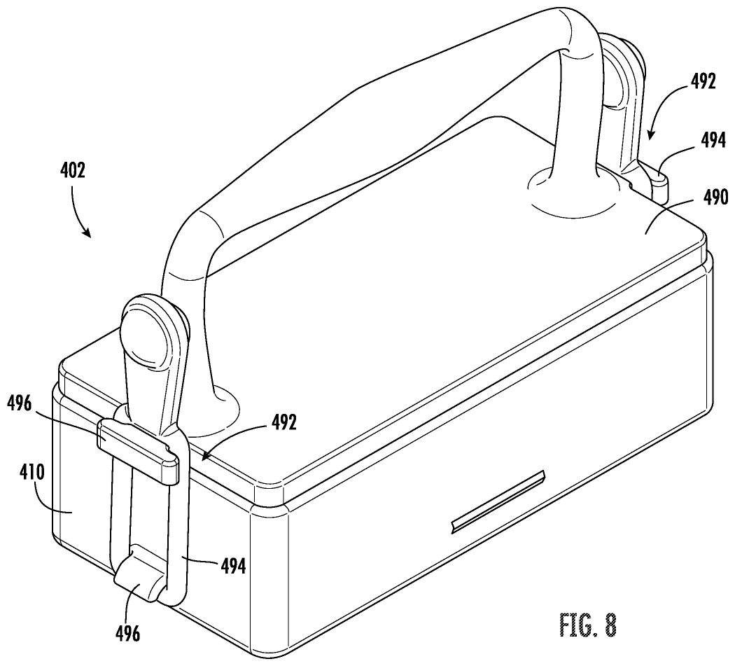

As noted above, it will be appreciated that the base of a transport and delivery system formed in accordance with various principles of the present disclosure may be considered a part of a material transport system with which the injectable material is transported to the patient. For example, in the example of an embodiment illustrated in FIG. 8, a material transport system 402 includes a base 410 in which may be formed a material transport device holder similar to the material transport device holder 112 within the base 110 of the transport and delivery system 100 illustrated in FIG. 2. The base 410 may be transported with a material transport device 140 such as described above with reference to FIG. 2. In the example of an embodiment illustrated in FIG. 8, a transport lid 490 is mated with the base 410 to close the material transport device 140 therein for safe transport. The transport lid 490 may be formed, at least in part, of a radioactive shielding material (e.g., tungsten. lead, or stainless steel), or may simply include a separate sheet of such material, such as a ⅛″ (0.3175 cm) sheet of stainless steel. A securement device 492 may be secured about the exterior of the base 410 and transport lid 490 to hold the transport lid 490 securely in place with respect to the base 410. In the example of an embodiment illustrated in FIG. 8, the securement device 492 includes elastomeric bands or loops 494 secured about projections or shoulders 496 on the base 410 and transport lid 490. It will be appreciated that other configurations of securement devices 492 are within the scope and spirit of the present disclosure, the specific configuration not being critical to the principles of the present disclosure. Once the material transport system 402 has been delivered to the location in the medical facility where the patient is to be administered the injectable material, the securement device 492 may be released (e.g., opened, loosened, removed, etc.) and the transport lid 490 may be removed from the base 410 to access the material transport device 140 therein. A cover such as the cover 320 of the transport and delivery system 300 illustrated in FIG. 7, and with features and structures of the cover 120 of the example of an embodiment illustrated in FIGS. 1, 2, and 4, may be mounted over and mated with the base 410 to deliver injectable material transported by the material transport device 440.

It will be appreciated that a transport and delivery system formed in accordance with any of the various principles of the present disclosure described above, in any combination thereof, may be well suited for delivery of injectable materials for imaging and diagnostics as well as for therapy and treatment. In particular, a transport and delivery system having one or more features described above may be suitable for delivery of therapeutic devices such as microspheres, and even more particularly, for delivery of radioactive microspheres.

Various structures and features of the embodiments described herein and illustrated in the figures have several separate and independent unique benefits. Therefore, the various structures and features described herein need not all be present in order to achieve at least some of the desired characteristics and/or benefits described herein. Moreover, the various features described herein may be used singly or in any combination. It will be appreciated that various features described with respect to one embodiment may be applied to another embodiment, whether or not explicitly indicated. Thus, it should be understood that one or more of the features described with reference to one embodiment can be combined with one or more of the features of any of the other embodiments described herein. That is, any of the features described herein can be mixed and matched to create hybrid designs, and such hybrid designs are within the scope of the present disclosure. Therefore, the present invention is not limited to only the embodiments specifically described herein. The above descriptions are of illustrative examples of embodiments only, and are not intended as limiting the broader aspects of the present disclosure. It will be appreciated that various features of each example of an embodiment described herein can be arranged and operate in substantially the same or similar manners in other of the examples of embodiments. Accordingly, for the sake of brevity and convenience, and without intent to limit, common elements with common functions may be indicated with the same reference characters differing in value by 100, reference being made to the above descriptions of similar elements and operations.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A transport and delivery system comprising:
a material transport device containing injectable material;
a base configured to hold said material transport device;
a material access device; and
a cover configured to hold said material access device;
wherein:
said cover is configured to be positioned over said base to mate said cover with said base to cause said material access device to align with said material transport device to access said material transport device.

2. The transport and delivery system of claim 2, wherein said cover includes a material delivery system configured to deliver the injectable material to a patient.

3. The transport and delivery system of claim 2, wherein said material delivery system comprises said material access device, and a delivery conduit system fluidly coupled with said material access device.

4. The transport and delivery system of claim 3, wherein:
said material access device comprises at least one needle; and
said delivery conduit system comprises at least one tubing.

5. The transport and delivery system of claim 4, wherein:
said at least one needle comprises an inlet needle and an outlet needle; and
said at least one tubing comprises inlet tubing fluidly coupled with said inlet needle, and outlet tubing coupled with said outlet needle.

6. The transport and delivery system of claim 5, wherein:
said material delivery system further comprises a priming cap defining a sealed chamber therein and configured to cover sharp ends of said inlet needle and said outlet needle; and
passing of flushing solution into said inlet tubing, through said inlet needle, through said sealed chamber, through said outlet needle, and through said outlet tubing results in a primed airless material delivery system primed for coupling with said base.

7. The transport and delivery system of claim 5, wherein said material transport device comprises a vial with a stopper defining a septum piercable by said at least one needle of said material access device as a result of mounting said cover on said base.

8. The transport and delivery system of claim 5, wherein:
said at least one tubing includes an outlet tubing fluidly coupled with said at least one needle to deliver the injectable material to a patient; and
said cover further comprises a mounting arm configured to maintain said outlet tubing oriented at an angle below horizontal.

9. The transport and delivery system of claim 1, wherein said base is configured to permit ejection of said material transport device therefrom.

10. An airless primed material delivery system comprising:
a material access device;
a material conduit system fluidly coupled with said material access device; and
a priming cap configured to cover said material access device and to maintain a sealed configuration of said material delivery system for priming.

11. The airless primed material delivery system of claim 10, wherein:
said material access device comprises at least one needle; and
said material conduit system comprises at least one tubing.

12. The airless primed material delivery system of claim 11, wherein:
said at least one needle comprises an inlet needle and an outlet needle;
said at least one tubing comprises inlet tubing fluidly coupled with said inlet needle, and outlet tubing coupled with said outlet needle;
said priming cap defines a sealed chamber therein configured to cover sharp ends of said inlet needle and said outlet needle; and
passing of flushing solution into said inlet tubing, through said inlet needle, through said sealed chamber, through said outlet needle, and through said outlet tubing results in a primed airless material delivery system.

13. The airless primed material delivery system of claim 12, further comprising a cover holding said material access device and said material conduit system, said cover including a mounting arm configured to maintain said outlet tubing oriented at an angle below horizontal.

14. The airless primed material delivery system of claim 10, further comprising a cover holding said material access device and said material conduit system, said cover being configured to align with a base holding a material transport device to align said material access device with the material transport device to deliver material from within the material transport device to a patient.

15. A method of transporting and delivering an injectable material, said method comprising:
delivering the injectable material in a material transport device; and
placing a cover over the base configured to hold the material transport device;
wherein the cover includes a material access device, so that upon mating the cover and the base, the material access device of the cover is aligned with the material delivery system held by the base to access the injectable material for delivery to a patient.

16. The method of claim 15, further comprising delivering the material delivery system separately from the base and cover.

17. The method of claim 16, further comprising ejecting the material delivery system from the base when delivery of the injectable material to the patient is complete.

18. The method of claim 15, wherein the cover further includes a material delivery system, said method further comprising priming said material delivery system to eliminate air within the material delivery system.

19. The method of claim 18, wherein:
the material access device comprises an inlet needle, an outlet needle, and a tubing system;
the material delivery system further comprises a priming cap;
sharp ends of the inlet needle and the outlet needle extend into a chamber within the priming cap;
said priming comprises flushing a material through the inlet needle, through the chamber within the priming cap, through the outlet needle, and through the tubing system to eliminate air from within the material delivery system; and
said method further comprises retaining the material within the tubing system and removing the priming cap before mating the cover with the base to allow the sharp ends of the inlet needle and the outlet needle to extend into the material transport device to access the injectable material for delivery to a patient.

20. The method of claim 15, further comprising delivering the material delivery system within the base to a medical facility at which the injectable material is to be delivered to the patient.
